(19) United States
(12) Patent Application Publication
(10) Pub. No.: US 2024/0389879 A1
(43) Pub. Date: Nov. 28, 2024

(54) BODY FAT SCALE

(71) Applicant: Guangdong Welland Technology Co.,Ltd., Zhongshan (CN)

(72) Inventor: RUIJUN GAO, Zhongshan (CN)

(73) Assignee: Guangdong Welland Technology Co.,Ltd., Zhongshan (CN)

(21) Appl. No.: 18/764,149

(22) Filed: Jul. 4, 2024

Related U.S. Application Data
(63) Continuation of application No. PCT/CN2023/099313, filed on Jun. 9, 2023.

(30) Foreign Application Priority Data
May 25, 2023 (CN) 2023106046084

(51) Int. Cl. A61B 5/0537

(52) U.S. Cl. CPC A61B 5/0537; A61B 2560/0431; A61B 2562/04

(57) ABSTRACT

The present invention discloses a body fat scale, including a scale body and a handle electrically connected to the scale body, where the handle includes a fixed base, a first telescopic base slidably disposed on one side of the fixed base, and a second telescopic base slidably disposed on the other side of the fixed base, and the first telescopic base and the second telescopic base may extend or retract along a length direction of the fixed base, to change a length of the handle. According to the above structure, the length of the handle can be adjusted according to a size of a palm of a user, so that the user can measure body fat at an optimal length, and the body fat and other parameters can be measured more accurately.

### CROSS-REFERENCE TO RELATED APPLICATIONS

The application claims priority to Chinese patent application No. 2023106046084, filed on May 25, 2023, the entire contents of which are incorporated herein by reference.

### TECHNICAL FIELD

The present invention relates to the field of body fat scales, and in particular, to a body fat scale.

### BACKGROUND

A body fat scale is a tool used for measuring body fat and other compositional parameters of a human body. In order to ensure that calculated body fat and other parameters are relatively accurate, the body fat scale generally has eight electrodes, including four electrodes distributed on a handle and four electrodes distributed on a scale body, which respectively measure impedance of upper and lower limbs of the human body. However, in an existing eight-electrode body fat scale, the length of the handle thereof cannot be adjusted, but sizes of palms of persons are different, especially the size of a palm of a child differs greatly from that of an adult. Therefore, it is difficult for the handle whose length cannot be adjusted to meet use requirements of different persons at the same time. If a difference between a product size and an actual required size is relatively large, an impact on measurement of the body fat is relatively large. Therefore, a body fat scale is urgently needed to solve the above problem.

### SUMMARY

The present invention aims to at least solve one of technical problems existing in the prior art. Therefore, the present invention provides a body fat scale.

A technical solution used to solve the technical problems in an embodiment of the present invention is as follows: a body fat scale includes a scale body and a handle electrically connected to the scale body, where the handle includes a fixed base, a first telescopic base slidably disposed on one side of the fixed base, and a second telescopic base slidably disposed on the other side of the fixed base, and the first telescopic base and the second telescopic base may extend or retract along a length direction of the fixed base, to change a length of the handle.

Further, the fixed base includes a first base body and a second base body;
the first telescopic base includes a first left housing and a second left housing, the first left housing is connected to one side of the first base body through a first sliding structure, and the second left housing is connected to one side of the second base body through a second sliding structure and is connected to the first left housing through a first detachable structure, so that the first left housing and the second left housing may extend or retract along the length direction of the fixed base; and
the second telescopic base includes a first right housing and a second right housing, the first right housing is connected to the other side of the first base body through a third sliding structure, and the second right housing is connected to the other side of the second base body through a fourth sliding structure and is connected to the first right housing through a second detachable structure, so that the first right housing and the second right housing may extend or retract along the length direction of the fixed base.

Further, the first base body and the second base body enclose to form a sliding cavity, and the first sliding structure includes:
a first sliding groove, formed in the first base body;
a first sliding block, disposed on the first left housing and extending into the first sliding groove; and
a first sliding base, disposed in the sliding cavity and connected to the first sliding block through a third detachable structure.

Further, a control plate is disposed in the sliding cavity, a first electrode sheet is disposed on the first left housing, a second electrode sheet is disposed on the second left housing, a third electrode sheet is disposed on the first right housing, and a fourth electrode sheet is disposed on the second right housing, and the control plate is electrically connected to the first electrode sheet, the second electrode sheet, the third electrode sheet, and the fourth electrode sheet.

Further, the third detachable structure includes a bolt, a threaded hole formed in the first sliding block, and a through hole formed in the first sliding base, and the bolt is in threaded connection with the threaded hole after passing through the through hole.

Further, the first telescopic base is connected to one side of the fixed base through a first locking structure, so that the first telescopic base may be kept in an extended state and a retracted state.

Further, the first locking structure includes a first clamping groove formed in the first telescopic base and a first hook and a second hook that are disposed on one side of the fixed base at intervals, and the first hook may be clamped into the first clamping groove when the first telescopic base is in the extended state, or the second hook may be clamped into the first clamping groove when the first telescopic base is in the retracted state.

Further, the first detachable structure and/or the second detachable structure are/is set as bolt connecting structures/a bolt connecting structure.

Further, the first detachable structure and/or the second detachable structure are/is set as clamping structures/a clamping structure.

Beneficial effects of the present invention: a body fat scale includes a scale body and a handle electrically connected to the scale body, where the handle includes a fixed base, a first telescopic base slidably disposed on one side of the fixed base, and a second telescopic base slidably disposed on the other side of the fixed base, and the first telescopic base and the second telescopic base may extend or retract along a length direction of the fixed base, to change a length of the handle. According to the above structure, the length of the handle can be adjusted according to a size of a palm of a user, so that the user can measure body fat at an optimal length, and the body fat and other parameters can be measured more accurately.

### BRIEF DESCRIPTION OF DRAWINGS

The above and/or additional aspects and advantages of the present invention will become apparent and easy to understand from the description of the embodiments in conjunction with the following accompanying drawings, where:

FIG. 1 is a schematic structural diagram in which a body fat scale is in a retracted state;

FIG. 2 is a schematic structural diagram in which a body fat scale is in an extended state;

FIG. 3 is an exploded view in which a body fat scale is in a retracted state; and

FIG. 4 is a schematic structural diagram of a first sliding base.

### DETAILED DESCRIPTION OF THE EMBODIMENTS

This part will describe the specific embodiments of the present invention in detail. The preferred embodiments of the present invention are shown in the accompanying drawings, and the function of the drawings is to supplement the description of the text part of the specification with graphics, which enables people to intuitively and vividly understand each technical feature and the overall technical solutions of the present invention, but cannot be understood as limiting the scope of protection of the present invention.

In the description of the present invention, “a plurality of” means more than two, “greater than a number”, “less than a number”, “exceed a number” and the like indicate that the number is excluded, and “above a number”, “below a number”, “within a number”, and the like indicate that the number is included. “First” and “second” are intended only for distinguishing between technical features but cannot be used to indicate or imply relative importance or implicitly specify a quantity of indicated technical features or implicitly specify a sequential relationship of indicated technical features.

In the description of the present invention, it should be understood that the positional descriptions referred to, for example, the directional or positional relationships indicated by up, down, front, rear, left, right, etc., are based on the directional or positional relationships shown in the drawings, and are only for convenience and simplification of description of the present invention, but not for indicating or implying that the referred device or element must have a specific direction, be constructed and operated in a specific direction, and thus should not be construed as limiting the present invention.

In the present invention, unless otherwise expressly defined, the terms such as “dispose”, “mount”, and “connect” should be understood in a broad sense. For example, it can be directly connected or indirectly connected through intermediate media; it can be in fixed connection, detachable connection, or integrated molding; it can be in mechanical connection; and it can be internal connectivity of two components or the interaction relationship between two components. For persons of ordinary skill in the art, specific meanings of the terms in the present invention may be appropriately determined with reference to the specific content in the technical solution.

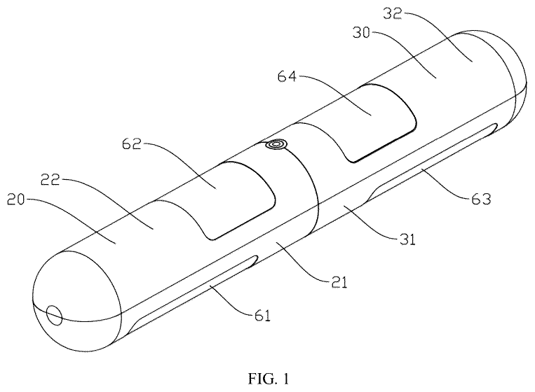

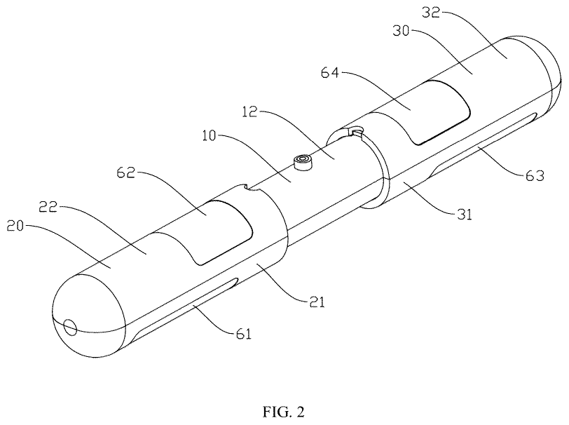

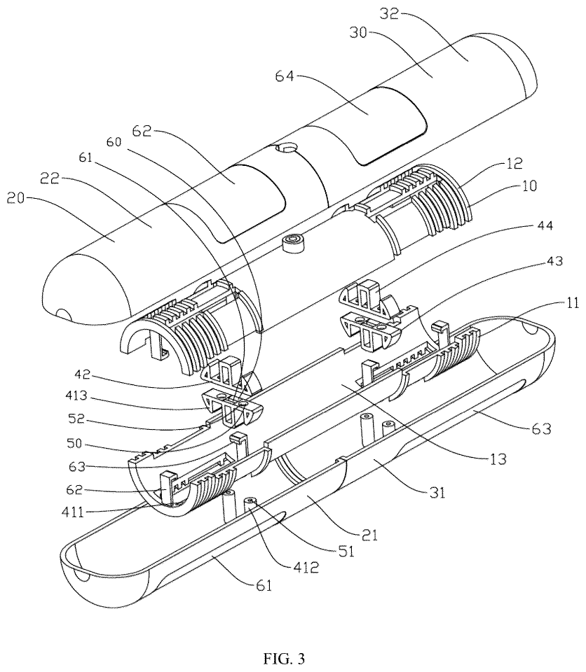

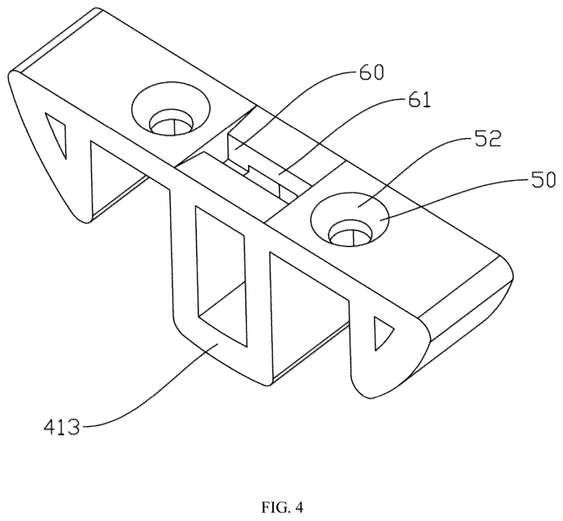

Referring to FIG. 1 to FIG. 4, a body fat scale includes a scale body and a handle electrically connected to the scale body, where the handle includes a fixed base 10, a first telescopic base 20 slidably disposed on one side of the fixed base 10, and a second telescopic base 30 slidably disposed on the other side of the fixed base 10, and the first telescopic base 20 and the second telescopic base 30 may extend or retract along a length direction of the fixed base 10, to change a length of the handle.

In the present invention, during assembly:
1. A first left housing 21 and a first right housing 31 are respectively placed on a first base body 11, a first sliding block 412 on the first left housing 21 is inserted into a first sliding groove 411 on one side of the first base body 11, and a third sliding block on the first right housing 31 is inserted into a third sliding groove on the other side of the first base body 11; then a first sliding base 413 on a first sliding structure 41 is placed in a sliding cavity 13, and a through hole 52 on the first sliding base 413 is opposite to a threaded hole 51 on the first sliding block 412; then a bolt is used to connect the first sliding base 413 and the first left housing 21 to one side of the first base body 11, a third sliding base on a third sliding structure 43 is placed in the sliding cavity 13, and a through hole on the third sliding base is opposite to a threaded hole on the third sliding block; and then the bolt is used to connect the third sliding base and the first right housing 31 to the other side of the first base body 11, thereby completing assembly of a first part.
2. A second left housing 22 and a second right housing 32 are respectively placed on a second base body 12, a sliding block on the second left housing 22 is inserted into a second sliding groove on one side of the second base body 12, and a fourth sliding block on the second right housing 32 is inserted into a fourth sliding groove on the other side of the second base body 12; then a second sliding base on a second sliding structure 42 is placed in the sliding cavity 13, and a through hole on the second sliding base is opposite to a threaded hole 51 on a second sliding block; then a bolt is used to connect the second sliding base and the second left housing 22 to one side of the second base body 12, a fourth sliding base on a fourth sliding structure 44 is placed in the sliding cavity 13, and a through hole on the fourth sliding base is opposite to a threaded hole on the fourth sliding block; and then the bolt is used to connect the fourth sliding base and the second right housing 32 to the other side of the second base body 12, thereby completing assembly of a second part.
3. The first part and the second part are assembled together. Specifically, the first left housing 21 is connected to the second left housing 22, and the first right housing 31 is connected to the second right housing 32, so that the first left housing 21 and the second left housing 22 are used as a whole to be extended and retracted, and the first right housing 31 and the second right housing 32 are used as a whole to be extended and retracted, and a connecting structure may be a bolt connecting structure, a clamping structure, or the like.
4. During use, a length of the handle is adjusted according to a requirement of a user. Specifically, when a child uses the body fat scale, a length of a required handle is relatively short due to a relatively small palm of the child, and therefore, the child may use the body fat scale when the handle is in a retracted state; and when an adult uses the body fat scale, a length of a required handle is relatively long due to a relatively large palm of the adult, and therefore, the first telescopic base 20 and the second telescopic base 30 may be pulled to enable the first telescopic base 20 and the second telescopic base 30 to slide relative to the fixed base 10, that is, the first telescopic base 20 extends toward a left side, and the second telescopic base 30 extends toward a right side, to increase the length of the handle. Certainly, sufficient lengths need to be reserved on connecting wires between a control plate and a first electrode sheet 61, a second electrode sheet 62, a third electrode sheet 63, and a fourth electrode sheet 64, to avoid that the connecting wires are pulled and damaged in an extending process.
5. An advantage of the present invention lies in that: according to the above structure, the length of the handle can be adjusted according to a size of a palm of a user, so that the user can measure body fat at an optimal length, and the body fat and other parameters can be measured more accurately.

Preferably, the fixed base 10 includes a first base body 11 and a second base body 12;
the first telescopic base 20 includes the first left housing 21 and the second left housing 22, the first left housing 21 is connected to one side of the first base body 11 through a first sliding structure 41, and the second left housing 22 is connected to one side of the second base body 12 through a second sliding structure 42 and is connected to the first left housing 21 through a first detachable structure, so that the first left housing 21 and the second left housing 22 may extend or retract along the length direction of the fixed base 10; and
the second telescopic base 30 includes the first right housing 31 and the second right housing 32, the first right housing 31 is connected to the other side of the first base body 11 through a third sliding structure 43, and the second right housing 32 is connected to the other side of the second base body 12 through a fourth sliding structure 44 and is connected to the first right housing 31 through a second detachable structure, so that the first right housing 31 and the second right housing 32 may extend or retract along the length direction of the fixed base 10.

The first base body 11 and the second base body 12 enclose to form the sliding cavity 13, and the first sliding structure 41 includes:
the first sliding groove 411, formed in the first base body 11;
the first sliding block 412, disposed on the first left housing 21 and extending into the first sliding groove 411; and
the first sliding base 413, disposed in the sliding cavity 13 and connected to the first sliding block 412 through a third detachable structure 50.

The control plate is disposed in the sliding cavity 13, the first electrode sheet 61 is disposed on the first left housing 21, the second electrode sheet 62 is disposed on the second left housing 22, the third electrode sheet 63 is disposed on the first right housing 31, and the fourth electrode sheet 64 is disposed on the second right housing 32, and the control plate is electrically connected to the first electrode sheet 61, the second electrode sheet 62, the third electrode sheet 63, and the fourth electrode sheet 64. Four electrode sheets are disposed to measure impedance of an upper part of a human body, and the body fat of the human body and other parameters are calculated in conjunction with impedance and other information of a lower part of the human body measured by the four electrode sheets on the scale body.

The third detachable structure 50 includes the bolt, the threaded hole 51 formed in the first sliding block 412, and the through hole 52 formed in the first sliding base 413, and the bolt is in threaded connection with the threaded hole 51 after passing through the through hole 52.

The first telescopic base 20 is connected to one side of the fixed base 10 through a first locking structure 70, so that the first telescopic base 20 may be kept in an extended state and a retracted state; preferably, the first locking structure 70 includes a first clamping groove 71 disposed on the first telescopic base 20 and a first hook 72 and a second hook 73 that are disposed on one side of the fixed base 10 at intervals, and the first hook 72 may be clamped into the first clamping groove 71 when the first telescopic base 20 is in the extended state, or the second hook 73 may be clamped into the first clamping groove 71 when the first telescopic base 20 is in the retracted state; and during sliding, each sliding base slides in the sliding cavity 13, and specifically, slides between the first hook 72 and the second hook 73, when the first telescopic base 20 is in the retracted state, the first clamping groove 71 is clamped to the second hook 73, to lock the retracted state, and when the first telescopic base 20 extends toward the left side, the first clamping groove 71 is detached from the second hook 73 first, and then moves to be clamped to the first hook 72, to lock the extended state.

The first detachable structure and/or the second detachable structure are/is set as bolt connecting structures/a bolt connecting structure or clamping structures/a clamping structure.

Certainly, the present invention is not limited to the above-mentioned implementation. Those skilled in the art may also make equivalent modifications or replacements without violating the spirit of the present invention. These equivalent modifications and replacements all fall within the scope of the claims of the present application.

What is claimed is:

1. A body fat scale, comprising a scale body and a handle electrically connected to the scale body, wherein the handle comprises a fixed base (10), a first telescopic base (20) slidably disposed on one side of the fixed base (10), and a second telescopic base (30) slidably disposed on the other side of the fixed base (10), and the first telescopic base (20) and the second telescopic base (30) can extend or retract along a length direction of the fixed base (10), to change a length of the handle;
the fixed base (10) comprises a first base body (11) and a second base body (12);
the first telescopic base (20) comprises a first left housing (21) and a second left housing (22), the first left housing (21) is connected to one side of the first base body (11) through a first sliding structure (41), and the second left housing (22) is connected to one side of the second base body (12) through a second sliding structure (42) and is connected to the first left housing (21) through a first detachable structure, so that the first left housing (21) and the second left housing (22) can extend or retract along the length direction of the fixed base (10);
the second telescopic base (30) comprises a first right housing (31) and a second right housing (32), the first right housing (31) is connected to the other side of the first base body (11) through a third sliding structure (43), and the second right housing (32) is connected to the other side of the second base body (12) through a fourth sliding structure (44) and is connected to the first right housing (31) through a second detachable structure, so that the first right housing (31) and the second right housing (32) can extend or retract along the length direction of the fixed base (10);
the first base body (11) and the second base body (12) enclose to form a sliding cavity (13), and the first sliding structure (41) comprises:
a first sliding groove (411), formed in the first base body (11);
a first sliding block (412), disposed on the first left housing (21) and extending into the first sliding groove (411); and
a first sliding base (413), disposed in the sliding cavity (13) and connected to the first sliding block (412) through a third detachable structure (50); and
a first electrode sheet (61) is disposed on the first left housing (21), a second electrode sheet (62) is disposed on the second left housing (22), a third electrode sheet (63) is disposed on the first right housing (31), and a fourth electrode sheet (64) is disposed on the second right housing (32).

2. The body fat scale according to claim 1, wherein a control plate is disposed in the sliding cavity (13), and the control plate is electrically connected to the first electrode sheet (61), the second electrode sheet (62), the third electrode sheet (63), and the fourth electrode sheet (64).

3. The body fat scale according to claim 1, wherein the third detachable structure (50) comprises a bolt, a threaded hole (51) formed in the first sliding block (412), and a through hole (52) formed in the first sliding base (413), and the bolt is in threaded connection with the threaded hole (51) after passing through the through hole (52).

4. The body fat scale according to claim 1, wherein the first telescopic base (20) is connected to one side of the fixed base (10) through a first locking structure (70), so that the first telescopic base (20) can be kept in an extended state and a retracted state.

5. The body fat scale according to claim 4, wherein the first locking structure (70) comprises a first clamping groove (71) formed in the first telescopic base (20) and a first hook (72) and a second hook (73) that are disposed on one side of the fixed base (10) at intervals, and the first hook (72) can be clamped into the first clamping groove (71) when the first telescopic base (20) is in the extended state, or the second hook (73) can be clamped into the first clamping groove (71) when the first telescopic base (20) is in the retracted state.

6. The body fat scale according to claim 1, wherein the first detachable structure and/or the second detachable structure are/is set as bolt connecting structures/a bolt connecting structure.

7. The body fat scale according to claim 1, wherein the first detachable structure and/or the second detachable structure are/is set as clamping structures/a clamping structure.
